(19) United States
(12) Patent Application Publication
(10) Pub. No.: US 2009/0005798 A1
(43) Pub. Date: Jan. 1, 2009

(54) Tool-Holder Instrument

(75) Inventor: Raphael Brunner, Biel (CH)

Correspondence Address: PEARNE & GORDON LLP, 1801 EAST 9TH STREET, SUITE 1200, CLEVELAND, OH, 44114-3108, US

(73) Assignee: BRUSO SARL, Brugg b/Biel (CH)

(21) Appl. No.: 11/916,085

(22) PCT Filed: Jun. 1, 2005
(86) PCT No.: PCT/CH2005/000309

§ 371 Date: Jun. 30, 2008

(51) Int. Cl. A61B 17/16

(52) U.S. Cl. 606/181

(57) ABSTRACT

The invention concerns a handle-shaped tool-holder instrument comprising a drive shaft (2) for a tool (7) and capable of being slid into the instrument (1) along the longitudinal axis from the rear of the instrument to be coupled with the tool-holding part (8) in a tool-holding head (4) at the front of the instrument, said tool-holding head Z(4) being mounted angularly steerable about a fixed axis (5) at the front of the instrument, means (3′, 9) being provided to lock the steerable head (4) in any selected angular position.

This invention concerns a tool-holder instrument in the form of a longitudinal handle, particularly for surgical applications, with a drive shaft for a tool that can be slid along a longitudinal axis into the instrument from the back of the instrument and connected to the tool-holder part of a rotary tool housed in a tool-holder head mounted on the front of the instrument.

There are many designs for tool-holder instruments, like for example, lancers, particularly used in orthopedic surgery. Some instruments have heads with which the tools (for drilling, burring, screwing, tapping, boring) are moved at a set angle in relation to the longitudinal axis of the instrument.

The goal of this invention is to provide a tool-holder instrument that can be used at various radii of action for work in a multitude of techniques, particularly orthopedic-type surgical techniques.

To reach this goal and to solve the problems related to a tool-holder instrument like the one described above, the instrument in this invention has the features in the characterizing part of claim 1.

Special preferred forms of embodiment of the invention are described in the subclaims.

The invention will be described in greater detail below using embodiments illustrated in the drawings:

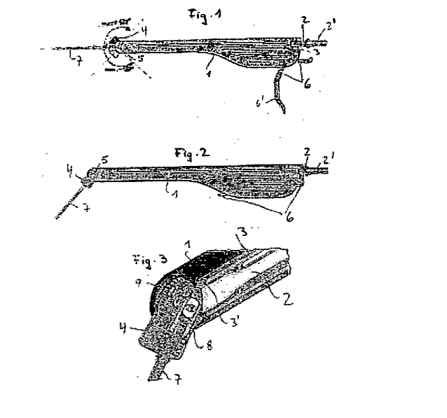

FIG. 1 shows a general view of the tool-holder instrument in the invention, with the drive and locking components in the unlocked position;

FIG. 2 shows a view similar to the one in FIG. 1 with the components in the locked position;

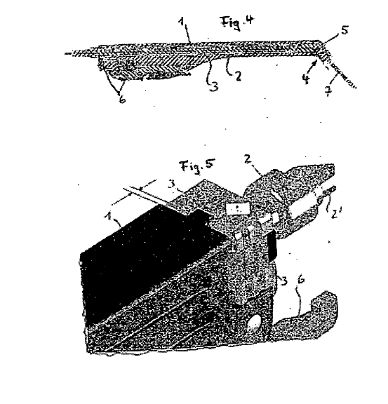

FIG. 3 shows a perspective view of a detail of the instrument (tool-holder head), particularly in cross section;

FIG. 4 shows the tool-holder instrument in a schematic section;

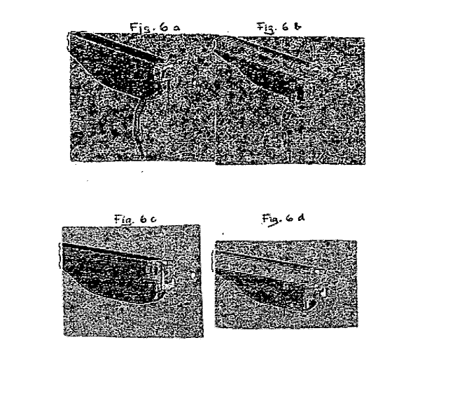

FIG. 5 shows an enlarged perspective view of another detail (the back part of the instrument with an articulated locking mechanism in the open position, and

FIG. 6a-6d shows the articulated locking mechanism, in partial cross section, in the open and closed positions, respectively.

The drawing in FIG. 1 shows a very schematic lateral view of the tool-holder instrument in the invention, comprising a case 1 in the form of a longitudinal handle that house inside a drive shaft 2 and a locking tube 3 (FIG. 3) that extend from the back along a longitudinal axis to the head of a tool-holder 4 in the front of the instrument.

The drive shaft 2 and the locking tube 3 can be taken off the instrument by pulling them out of the case toward the back or can be locked in the working position using an articulated mechanism 6 (see details in FIGS. 6a-6d).

The tool-holder head 4 can be moved at an angle around a set axis 5 of ±180° at the maximum from the neutral position 0° (directed along the longitudinal axis of the instrument) and is locked in any position chosen by means of said locking tube 3 (see detail in FIG. 3).

FIG. 2 shows the instrument with the drive and locking components, 2 and 3 respectively, locked in the longitudinal direction by the articulated mechanism 6 with the tool-holder head equipped with a tool (drill) 7.

The drive shaft 2, which can be connected in back 2′ to a motor (not shown) is connected in the working position in front to a tool-holder part 8 for a rotary tool 7 housed in the head of the tool-holder 4.

The structure of the coupling between the drive shaft 2 and said tool-holder part 8 can be any type, for example the universal joint type, a Cardan joint, a toothed wheel or another. The structure of the coupling mechanism that allows it to move ±180° will be described in a separate patent and is not part of this invention.

The locking tube 3 in the working position (locking the tool-holder head 4) engages in a toothed part 9 on the tool-holder head 4 and holds said head in the position chosen before it was locked.

The drilling instrument gear with a head that can be moved at an angle, when connected to a motor (Stryker, Hudson, AO or other coupling) allows the transmission of torque. Connecting different ancillary devices 7 to the front of its moveable head 4 allows it to drill, burr, screw and bore different parts of bones during orthopedic-type surgical procedures. When a screwdriver is connected to the instrument, the system allows various implantable or non-implantable screws to be screwed in, such as hex, torx, Phillips head or other screws.

The main innovation lies in the fact that the tool-holder head 4 can be moved and can cover a radius of action ranging theoretically from +180° to −180° going through 0°.

This makes it possible to cover a radius of action that fixed offset angle drills (ex. 45°) cannot guarantee. For this reason, it can be used in a multitude of orthopedic-type surgical techniques covering traumatology and surgery on the knee, spinal column, shoulder, limbs or maxillo-facial surgery, where the anatomy of the bones requires different approaches or access.

Due to its design, the proximal part of the instrument takes up little space, allowing minimally invasive techniques.

The fact that the head 4 of the instrument can be moved thus makes it possible to reach certain bony cavities or places difficult to access that could be reached only by several traditional instruments combined, for example, in traumatology, setting fractures by inserting plates, which traditionally can combine the use of various guides and flexible hoses to drill and thread passages for screws. In some cases, the instrument can replace this combination of two instruments, thus facilitating the technique.

Indexing the position of the head is done by the simple motion of lowering the lever 6′. This frees the articulated part and makes it possible to withdraw the tube 3. This frees the teeth 9 of the head 4 which, in this way, can be * moved at an angle chosen by the practitioner for the particular surgical application chosen. Once the angle is chosen, the tube 3 is pushed in order to lock the teeth 9 on the head 4 in position. The lever 6′ must be put into the closed position so that it transmits constant pressure on the articulated part, which itself, by leaning on the tube 3, locks the head 4 in position. In this position, the instrument can be used in rotation at the chosen angle.

The instrument can also be completely dismantled for deep cleaning before sterilization. The mechanism used to dismantle the instrument is the same as the one that makes it possible to move and lock the position of the angle of the head 4. The mechanism for dismantling the instrument is very simple to use and takes a minimum of movement to perform the dismantling operation. One need only lower the lever 6′, which frees the articulated part and position the head at 0°, then remove the drive shaft 2 and the tube 3. Once the device is dismantled, the user will have three pieces of the instrument (excluding ancillary devices), i.e.: the main body 1, the drive shaft 2 and the tube 3. These three separate elements effectively meet the requirements and directives for decontaminating, cleaning and sterilizing surgical instruments. The instrument is reassembled in reverse order from the operations above.

The shape and the lateral grooves on the case 1 have been designed to allow the attachment of guides to adapt and use the product in surgical techniques assisted by navigation. These grooves also make it possible to attach a sliding lateral handle for added stability when the instrument is used.

1. A tool-holder instrument in the form of a longitudinal handle, particularly for surgical applications, with a drive shaft (2) for a tool (7), which shaft (2) can be slid along a longitudinal axis in the instrument from the back of the instrument and be connected to the tool-holder part (8) of a rotary tool housed in a tool-holder head (4) mounted on the front of the instrument, characterized by the fact that the tool-holder head (4) housing said tool-holder part (8) is mounted on the front of the instrument in such a way that it can be moved at an angle in relation to the longitudinal axis of the instrument around a set swiveling axis transverse to that axis; means (3′, 9) are provided to lock the moveable head into any angular position chosen.

2. The instrument in claim 1, characterized by the fact that the means of locking (3′, 9) the moveable head (4) include a tube (3) to be inserted into the instrument from the back of the instrument surrounding the drive shaft (2) with play allowing free relative movement between these two elements; the front edge (3′) of said tube (3) can be brought into contact with a part (9) of the head (4) to lock the head in a pre-selected position.

3. The instrument in claims 1 and 2, characterized by means (6) of locking said tube (3) and the drive shaft (2) against axial displacement in their working positions, that is, in locking contact with the head (4), respectively connected to said tool-holder part (8) to drive the latter.

4. The instrument in claim 3, characterized by the fact that the locking means (6) include an articulated mechanism whose stopping elements in the locking position are applied against the back of said tube (3) and, if need be, against the back of the drive shaft.

5. The instrument in one of claims 1-4, characterized by the fact that the coupling between the drive shaft (2) and said tool-holder part (8) in the tool-holder head (4) is such that these two components (2, 8) can only be coupled and uncoupled in the position where the components (2, 3) are aligned on said longitudinal axis of the instrument, i.e., in the 0° angle position of the tool-holder head.

6. The instrument in one of claims 1-5, characterized by the fact that the drive components (2) and locking components (3) can be assembled and disassembled individually, particularly for cleaning and sterilizing them.

7. The instrument in one of claims 1-6, characterized by the fact that the tool-holder head (4) can be moved by an angle up to ±180° depending on the coupling mechanics between said tool-holder part (8) and the drive shaft (2).

8. The use of the instruments in claims 1-7 for surgical applications.
